(19) United States
(12) Patent Application Publication
(10) Pub. No.: US 2011/0087036 A1
(43) Pub. Date: Apr. 14, 2011

(54) PROCESS FOR PREPARING DIHYDROPYRAN AND ITS USE AS PROTECTIVE AGENT

(76) Inventor: Niklaus Künzle, Ausserberg (CH)

(21) Appl. No.: 12/900,920

(22) Filed: Oct. 8, 2010

Related U.S. Application Data
(60) Provisional application No. 61/250,248, filed on Oct. 9, 2009.

(51) Int. Cl. C07D 309/18

(52) U.S. Cl. 549/356

(57) ABSTRACT

A process for the protection of a compound having at least one reactive functional group selected from hydroxy, mercapto, carboxyl, amino and amide, with 3,4-dihydro-2H-pyran (DHP), wherein said DHP is obtained by contacting tetrahydrofurfuryl alcohol (THFA) entrained in a carrier gas with a catalyst comprising aluminium oxide (Al₂O₃), and wherein the carrier gas is water vapor.

### CROSS-REFERENCE TO RELATED APPLICATIONS AND CLAIM OF PRIORITY

This application claims the benefit of priority from U.S. Provisional Patent Application No. 61/250,248 filed Oct. 9, 2009, the disclosure of which is incorporated herein by reference.

### BACKGROUND OF THE INVENTION

A process for the protection of a compound having at least one reactive functional group selected from hydroxy, mercapto, carboxy, amino and amido, with 3,4-dihydro-2H-pyran (DHP), characterized in that said DHP is obtained by, wherein said DHP is obtained by a novel process.

3,4-dihydro-2H-pyran is an important reagent and building block in the synthesis of pharmaceutical and agrochemical compounds, see for example U.S. Pat. No. 5,536,734. Several processes for the preparation of DHP are described in the prior art.

U.S. Pat. No. 2,976,299 discloses a process for producing DHP by passing vaporized tetrahydrofurfuryl alcohol (THFA, CAS [97-77-4]) through a catalyst comprising Al₂O₃ and TiO₂. The vaporized THFA is entrained in a carrier gas such as nitrogen or hydrogen.

U.S. Pat. No. 3,518,281 discloses a process for producing DHP by conversion of vaporized THFA at a temperature from 200 to 375° C. using η-Al₂O₃ as a catalyst. As above, the THFA may be entrained in a carrier gas such as nitrogen or argon when contacted with the catalyst.

If gases such as nitrogen are used, however, considerable amounts of product are lost by removal with the carrier gas, as condensation is usually not sufficient for completely separating the products from the carried gas. Moreover, the above processes suffer from the drawback that they require frequent regeneration of the catalyst resulting in interruption of the production. Since the regeneration is typically carried out at elevated temperatures in the presence of air or oxygen while the catalyst is still installed.

One object of the present invention is to provide a simple and cost efficient process for the production of DHP, wherein DHP is obtained in high yields and good recovery rates, with low amounts of by-products, and wherein the used catalyst has a long life and little regeneration is required. A further object of the present invention is to use said high quality DHP with low amounts of by-products as a protecting agent for the protection of reactive functional groups.

### SUMMARY OF THE INVENTION

These objects have been achieved by the present invention, which provides a process for producing 3,4-dihydro-2H-pyran (DHP), comprising contacting tetrahydrofurfuryl alcohol (THFA) entrained in a carrier gas with a catalyst comprising aluminium oxide (Al₂O₃), wherein the carrier gas comprises water vapor, and wherein the thus obtained DHP has a very low content of by-products, especially a low acrolein amount.

Scheme 1 below shows the conversion of THFA (formula I) in the presence of a solid phase Al₂O₃ catalyst to form DHP (formula II) and H₂O, which typically takes place at elevated temperatures.

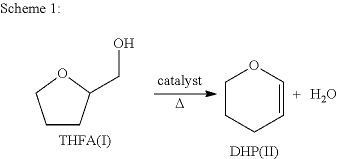

Since long, DHP is well known as a protecting agent for protecting compounds having reactive functional groups. See for example Woods, G. F. et al, J. Am. Chem. Soc. 1947, 69, 2246. The obtained ethers are relatively stable against under basic conditions as well as against compounds such as alkali, organolithium and Grignard reagents, Red-Al®, lithium aluminum hydride, carboxylic acid anhydrides, and in chromium trioxide oxidations. The alcohol can be released easily by mild acidic hydrolysis with diluted acids.

It is also possible to stabilize Poly(oxymethylene) polymers with 3,4-dihydro-2H-pyran against basic media by blocking terminal hydroxy groups. References are for example the “Technical Information Bulletin Number AL-106” of Aldrich Chemical Company, Inc., May 1993 and Greene, T. W. in Green's PROTECTIVE GROUPS IN ORGANIC CHEMISTRY, Fourth edition, Wiley, 2007, which both are herein included by reference.

Claimed is a process for the protection of a compound having at least one reactive functional group selected from hydroxy, mercapto, carboxy, amino and amido, with 3,4-dihydro-2H-pyran (DHP), characterized in that said DHP is obtained by contacting tetrahydrofurfuryl alcohol (THFA) entrained in a carrier gas with a catalyst comprising aluminium oxide (Al₂O₃), and wherein the carrier gas is water vapor. According to the description below it is clear that some one or more additional purge gas(es) can be present in the process for preparing DHP, while water vapor is always the main carrier gas.

Because of the very low amount especially of toxic by-products, DHP obtained by the above mentioned process, i.e. using water vapor as the main carrier gas, can be advantageously used as protecting agent for reactive groups compared to DHP obtained by conventional processes.

One of the major impurities while producing DHP is acrolein. Acrolein is a clear, colorless to straw-colored liquid with a pungent, suffocating odor. It is volatile and produces toxic concentrations even at room temperature. Vapors may travel to a source of ignition and flash back. The odor of acrolein may not provide an adequate warning of hazardous concentrations. Acrolein is toxic by all exposure routes, especially very toxic when inhaled. Exposure causes inflammation and irritation of the skin, respiratory tract, and mucous membranes. Delayed pulmonary edema may occur after inhalation. Systemic effects may occur after exposure by any route. Acrolein concentrations in the gaseous phase of 2 ppm are immediately harmful according to the classification of the National Institute for Occupational Safety and Health: Immediately dangerous to life or health (NIOSH IDLH). Thus, for a secure use on DHP low levels especially of acrolein is desired. In DHP obtained according to the present process acrolein can be reduced to about 20 ppm in DHP or lower. Thus, acrolein content in the gaseous phase above a reaction mixture will be much lower then 2 ppm, even at room temperature.

The underlying process for obtaining DHP is described in detail further below and is also further below illustrated in FIG. 1 and FIG. 2, as well as in Example 1. In a preferred embodiment DHP can be used as protective agent both, for discrete molecules or for protection of oligomeric or polymeric compounds comprising a distribution of similar compounds of different chain length. According to the invention the oligomeric or polymeric compounds needs to comprise at least one type of the reactive groups as described above for discrete compounds. Thus, one single molecule of an oligomeric or polymeric compound might comprise more then one reactive groups which can be protected by DHP. In the case of polymeric compounds both solubility and sensitivity towards bases can be controlled by the degree of derivatisation of the reactive groups with DHP.

In the case of discrete compounds the above process can be specified in that the compound having at least one reactive functional group is of formula

H—X III,

wherein

X is selected from —OR¹, —SR¹, and —OC(═O)R¹, wherein R¹ is a substituent selected from the group consisting of C₁₋₁₀-alkyl, C₂₋₁₁-acyl, C₃₋₈-cycloalkyl, C₄₋₉-cycloalkanoyl, aryl, aralkyl, aroyl, hetaryl and hetaroyl, or

X is —NR²R³, wherein R² and R³ independently are selected from the group consisting of hydrogen, C₁₋₁₀-alkyl, C₂₋₁₁-acyl, C₃₋₈-cycloalkyl, C₄₋₉-cycloalkanoyl, aryl, aralkyl, aroyl, hetaryl and hetaroyl, with the proviso that at least one of R² and R³ is different from hydrogen, and wherein the reaction product is of formula

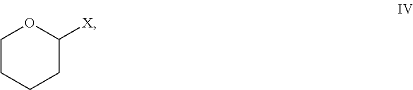

wherein X is as defined above.

As an example formation of the respective tetrahydropyranyl derivative of formula III is depicted in the first step of Scheme 2 below in the case that compound of formula IV is an alcohol.

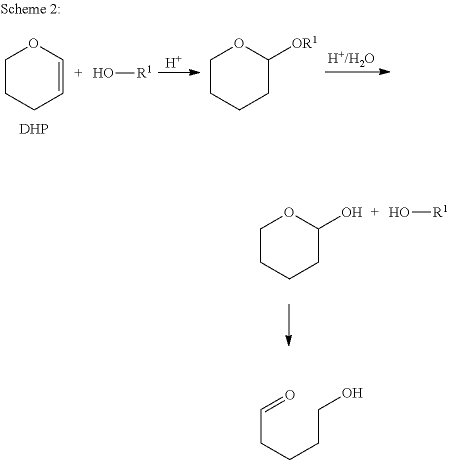

When the compound of formula III is formed, chemical reactions in non-acidic medium can be carried out at the residue R before the compound of formula III is treated with an aqueous acid to restore the alcohol from protection. Thus, the restored alcohol will be not identical to the alcohol used for the formation of the tetrahydropyranyl derivative of formula III.

The base tolerant tetrahydropyranyl derivatives of formula, compound having a tetrahydropyranyl protecting group. According to Scheme 2, finally, acidic hydrolysis leads to restoration of the alcohol (HO—R) and ring opening to give tetrahydropyranyl alcohol, which thus leads to formation of 5-hydroxypentanal.

Accordingly to Scheme 2 where the compound of formula III is a thiol (R—SH), a carboxylic acid (RCOOH), an amine (R₂NH or RNH₂), or an amide ((RCO)₂NH or RCONH₂), the respective mercapto, carboxy, amino or amido group is restored under ring opening and formation of 5-hydroxypentanal under mild acidic conditions.

In the definitions below, generally Cₙ₋ₘ indicates a group comprising n to m carbon atoms. For example C₁₋₄ and C₂₋₁₁ refer to an alkyl group having 1 to 4, or 2 to 11 carbon atoms, respectively.

C₁₋₁₀-Alkyl refers to a linear or branched alkyl group having 1 to 10 carbon atoms, optionally substituted with one or more halogen atoms. Halogen may be F, Cl, Br and I. Examples of C₁₋₁₀-alkyl are methyl, ethyl, propyl, isopropyl, butyl, i.e. n-butyl, sec-butyl or tert-butyl, pentyl, hexyl, heptyl, octyl, nonyl, decyl, bromomethyl, chloromethyl, fluoromethyl, dibromomethyl, dichloromethyl, difluoromethyl, up to perfluoroalykl, such as perfluoromethyl or perfluoro-ethyl. Preferably used as C₁₋₁₀-alkyl is C₁₋₄-alkyl, i.e. methyl, ethyl, propyl, isopropyl, butyl, i.e. n-butyl, sec-butyl, tert-butyl, bromomethyl, chloromethyl, fluoromethyl, dibromomethyl, dichloromethyl, difluoromethyl, perfluoromethyl or perfluoroethyl.

C₁₋₄-Alkoxy refers to a C₁₋₄-alkyl group as defined above, which is attached via an oxygen atom. C₁₋₄-Alkoxy is selected from methoxy, ethoxy, propoxy, isopropoxy, butoxy, i.e. n-butoxy, sec-butoxy or tert-butoxy.

C₂₋₁₁-Alkanoyl refers to a linear or branched C₂₋₁₁-alkyl group as defined above, which is attached via a carbonyl group, for example acetyl, propionyl, butyryl, pentylcarbonyl, hexyl-carbonyl, heptylcarbonyl, octylcarbonyl, nonanylcarbonyl, decylcarbonyl, 2-bromoacetyl, 2-chloroacetyl, 2-fluoroacetyl, dibromoacetyl, dichloroacetyl, difluoroacetyl, tribromoacetyl, trichloroacetyl, trifluoroacetyl, 2-bromopropionyl, 3-bromopropionyl, 2-chloropropionyl, 3-chloropropionyl, 2-fluoropropionyl, 3-fluoropropionyl, 2,2-dibromopropionyl, 2,3-dibromopropionyl, 2,2-dichloropropionyl, 2,3-dichloropropionyl, 2,2-difluoropropionyl, 2,3-difluoropropionyl, perbromopropionyl, perchloropropionyl or perfluoropropionyl.

C₃₋₈-Cycloalkyl refers to a C₃₋₈-cycloalkyl group, either unsubstituted or substituted by one or more halogen atoms. Halogen may be F, Cl, Br and I. C₃₋₈-cycloalkyl is for example cyclopropyl, cyclobutyl, cyclopentyl, cyclohexyl, cycloheptyl or cyclooctyl.

C₄₋₉-Cycloalkanoyl refers to a C₃₋₈-cycloalkyl group, as which is attached via a carbonyl group.

Aryl means phenyl or naphthyl, optionally substituted with one to three substituents selected from halogen atoms, C₁₋₄-alkyl as defined above, C₁₋₄-alkoxy as defined above, cyano and nitro groups. Halogen may be F, Cl, Br and I. Examples of aryl are phenyl, 4-halophenyl, 3,4-dihalo-phenyl, 2,5-dihalophenyl, 4-C₁₋₄-alkyl-phenyl, 3,4-di-C₁₋₄-alkyl-phenyl or 3,5-di-C₁₋₄-alkyl-phenyl.

Aralkyl refers to an aryl group as defined above, which is attached via a methylene group. Examples for aralkyl are benzyl, 2-phenethyl, 2-methylbenzyl, 3-methylbenzyl, 4-methyl-benzyl, 2,4-dimethylbenzyl, 2-(4-ethylphenyl)ethyl, naphthylmethyl or naphthylmethyl.

Aroyl refers to aryl as defined above, via a carbonyl group. Aroyl is for example benzoyl, halobenzoyl such as 4-bromobenzoyl or 4-chlorobenzyloxy, naphthalene-1-carbonyl, or halonaphthalene-1-carbonyl.

Hetaryl means a 5- or 6-membered heterocyclic aromatic group having at least one, up to three at the most, ring oxygen, nitrogen or sulfur atoms in the ring. The ring may be either unsubstituted or substituted by one or more halogen atoms or one or more C₁₋₄-alkyl groups as defined above. Halogen may be F, Cl, Br and I. Examples of hetaryl are 2-furanyl, 3-furanyl, 2-thienyl, 3-thienyl, 2-benzo[b]furanyl, 2-benzo[b]thienyl, 2-pyridinyl, 3-pyridinyl or 4-pyridinyl.

Hetaroyl means hetaryl as defined above, which is attached via a carbonyl group. Suitable hetaroyl groups are for example 2- or 3-thenoyl, nicotinoyl, 2- or 3-furoyl.

In the following the present process to obtain DHP with low amounts of by-products, especially of acrolein, is shown in figures and also explained.

### DESCRIPTION OF THE FIGURES

FIG. 1 shows a simple schematic representation of the production process of DHP according to the present invention with subsequent distillation. The number of heat exchangers and vaporizers before the reactor are dependent from the conditions in the industrial environment.

Water (14) is pumped through heat exchanger (1) and heated by the raw product stream leaving the reactor (79. After leaving the heat exchanger (1) the water has a temperature of about 195 to 210° C. The water vapor then is passed through vaporizers (2) and (3) to reach a temperature of about 370 to 395° C.

THFA (15) is pumped through heat exchanger (4) and vaporizer (5) to reach a temperature of about 300° C. Gaseous THFA is entrained in the water vapor in a mixing device (6) and passed through reactor (7) which is the Al₂O₃-comprising catalyst. The temperature in the reactor is about 350° C. at the beginning of the production run and is further maintained at about 330° C. The reactor pressure is maintained at atmospheric pressure. A small amount of nitrogen can be fed into the system, advantageously between heat exchanger (1) and vaporizer (2).

The obtained crude product is cooled with the feed water (14) at heat exchanger (1) and nitrogen and other uncondensable gases were removed in a separator (not shown). The crude product (16), mainly consisting of DHP and water, is further passed into the decanter (8) and separated from water. The water was disposed (17) as it contains at room temperature only about 1.6 weight % of DHP. Following separation from water, the crude product optionally is collected in one or more tanks (9) before subjected to distillation.

For a batch distillation the crude product is filled into a distillation boiler (10) and heated to reflux over a column (11), said column optionally is packed with structured bodies. Preferably, the distillation is carried also out at atmospheric pressure. The sump (18) is periodically removed from the distillation boiler (10).

At the beginning, the reflux from the column is returned completely to the column after passing a condenser (12). After reaching steady state a constant flow of the reflux is passed through the decanter (13), which is filled with water to wash the reflux, before returning the washed reflux to the column. Azeotropes remain in the decanter and the water (19) in the decanter is replaced periodically until the organic phase meets the required specifications, particularly until the acrolein content is 20 ppm or less. Then the reflux ratio is changed and the product DHP (20) is distilled into suitable tank(s). Expediently, the distillation is interrupted when the temperature in the distillation boiler drops to 130° C. or less.

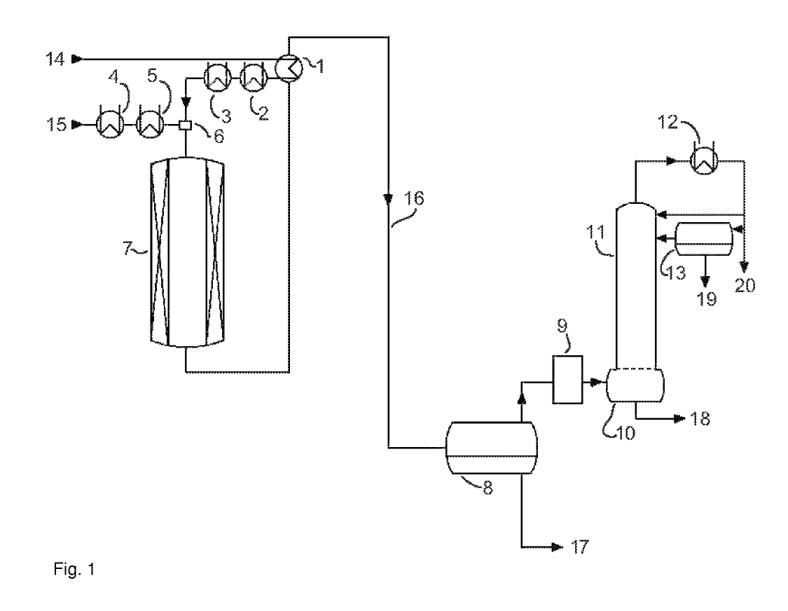

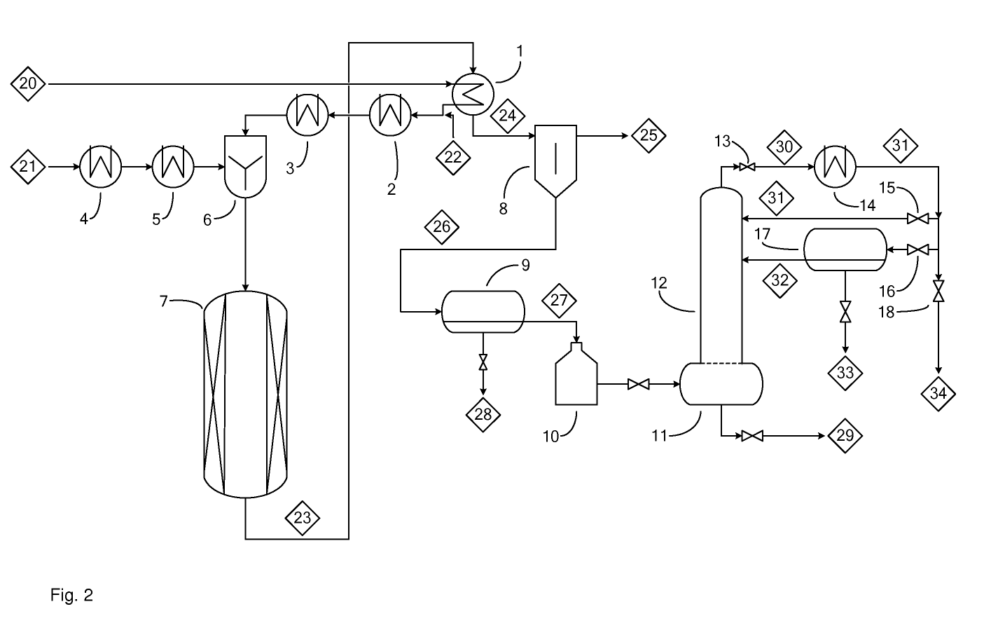

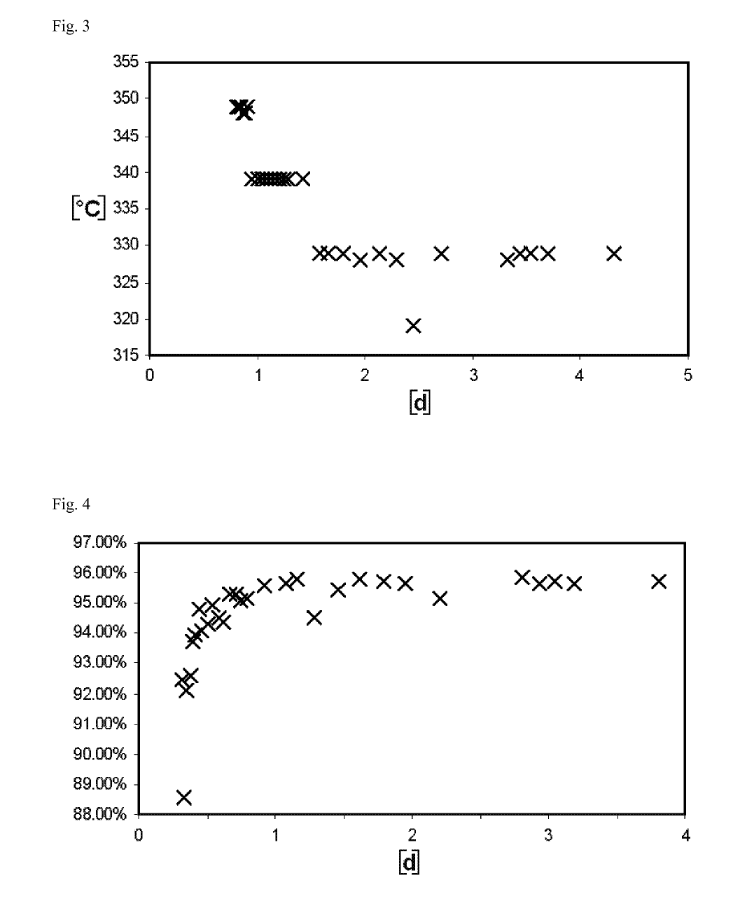

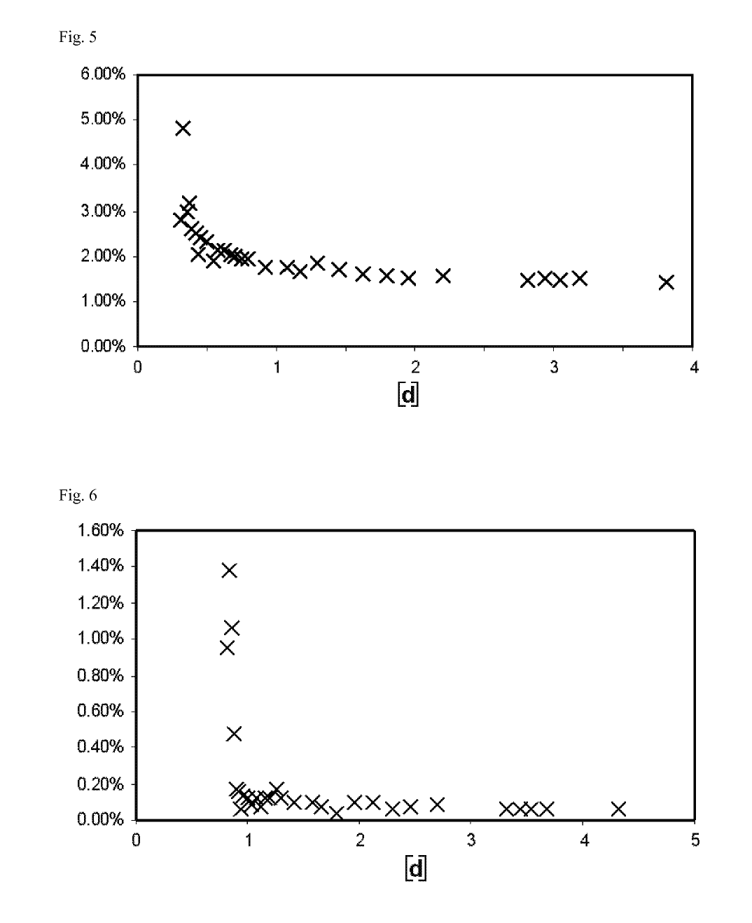

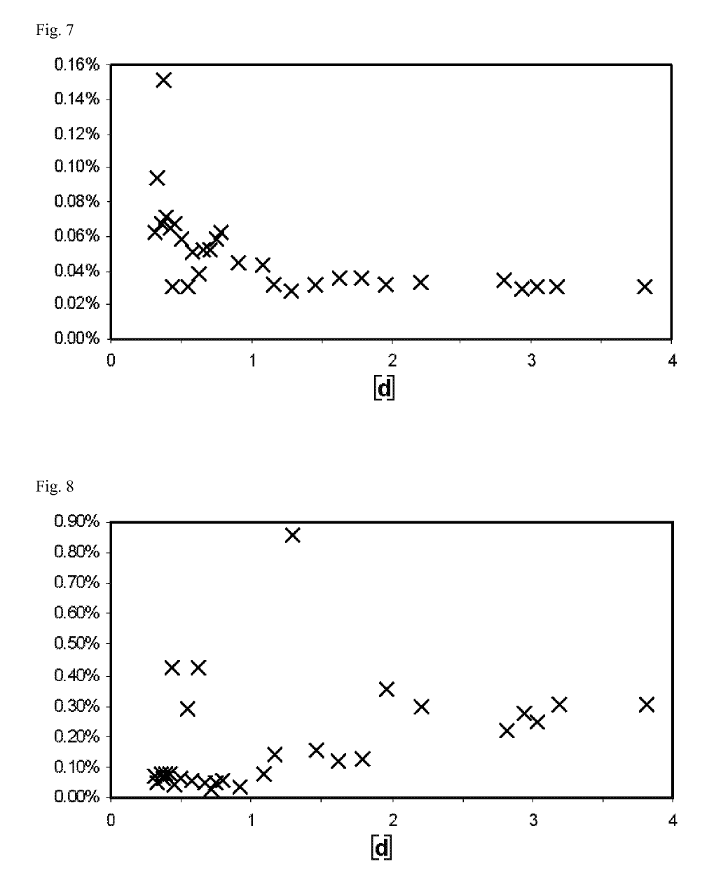

Listing of Embodiments of FIG. 1
1: heat exchanger
2: vaporizer
3: vaporizer
4: heat exchanger
5: vaporizer
6: mixing device
7: reactor
8: decanter
9: tank(s)
10: distillation boiler
11: distillation column
12: condenser
13: decanter
14: water feed
15: THFA feed
16: crude product
17: water
18: sump
19: water
20: product (DHP)

FIG. 2 shows a more detailed schematic representation then FIG. 1 of the production process of DHP according to the present invention with subsequent distillation. The number of heat exchangers and vaporizers before the reactor are dependent from the conditions in the industrial environment. Construction elements are marked with plain numbers (1 to 18). Flows are marked with numbers in diamond fields (20 to 34). Only valves necessary for an improved distillation circuit and obtaining the product (13, 15, 16 and 18) are numbered to allow simple identification in the description.

Water (20) is fed through a heat exchanger (1) and preheated by the crude reaction mixture (23) leaving the reactor (7). After leaving the heat exchanger (1) the water vapor (approx. 195 to 210° C.) is passed through one or more vaporizer(s) (2, 3) to reach about 370 to 395° C.

THFA (21) is fed through a heat exchanger (4) and a vaporizer (5) to reach about 300° C. Gaseous THFA is entrained in the water vapor in a mixing device (6) and passed through a reactor (7) charged with the Al₂O₃-comprising catalyst. Starting temperature in the reactor is about 350° C. and is usually further maintained at approx. 330° C. The reactor can be operated at atmospheric pressure. A small amount of an additional purge gas (22) might be fed into the system, preferably between heat exchanger (1) and vaporizer (2).

After leaving the reactor (7), the crude reaction mixture (23) is condensed in the heat exchanger (1), and the condensed product (24) is fed to a gas-liquid separator (8), where gaseous components (25), i.e. purge gas(es) and/or other uncondensable gas(es), are removed. The crude condensate (26), mainly consisting of DHP and water, then is passed into the decanter (9) and separated from water (28), which can be disposed as it contains at room temperature only about 1.6 weight % of DHP.

An azeotropic distillation of the crude product (27) can be carried out in continuous mode or in batch mode. In continuous mode the crude product (27) leaving the decanter (9) is passed continuously into the distillation boiler (11), whereas in batch mode the crude product is first collected for example in one or more tanks (10) and then passed into the distillation boiler (11). Expediently, the distillation is carried out in batch mode to obtain DHP with very low acrolein amounts. Thus, the following description only refers to a batch mode distillation.

In the distillation boiler (11) the crude product (27) is heated to reflux over a column (12), said column (12) might optionally be operated with separation plates or filled with a structured packing. Expediently, distillation is carried out at atmospheric pressure. The sump (29) can be periodically removed.

Regulated by a valve (13) the head product (30) is passed through a condenser (14) to obtain a condensed head product (31). The first runnings of the condensed head product (31) are returned completely to the column. After reaching steady state, a more or less constant part of the condensed head product (31), i.e. between 50 and 100% of the regulated by valves (15) and (16), is fed to a decanter (17) which is charged with water to wash the condensed head product (31) and to remove acrolein. Washed head product (32) is then returned to the column (12) as well as any condensed head product (31) passing valve (15). Expediently, about ⅔ of the condensed head product (31) is passed through said decanter (17) where azeotropes comprised in the head product remain in the decanter. The water (33) in the decanter is replaced at appropriate intervals until the organic phase in the decanter (i.e. washed head product (32)) meets the required specifications, particularly until the acrolein content is 20 ppm or less. Distilled product (34) can be removed from the circuit via valve 18. In a preferred embodiment to remove the product (DHP, 34) the ratio of the total reflux (i.e., condensed head product (31) which is fed back to the column either after passing valves (15) or (16)) to removed DHP (34) is controlled to be about ¾ and of about ¼ of the condensed head product (31), respectively. Removed DHP preferably is stored in a suitable tank. The distillation should be interrupted when the temperature in the distillation boiler drops to about 130° C. or less.

Listing of Embodiments of FIG. 2

### Numbers in Brackets Indicate the Corresponding Embodiment in FIG. 1

| | | |
|---|---|---|
| 1: | heat exchanger | (1) |
| 2: | vaporizer | (2) |
| 3: | vaporizer | (3) |
| 4: | heat exchanger | (4) |
| 5: | vaporizer | (5) |
| 6: | mixing device | (6) |
| 7: | reactor | (7) |
| 8: | gas-liquid separator | (not shown) |
| 9: | decanter | (8) |
| 10: | tank(s) | (9) |
| 11: | distillation boiler | (10) |
| 12: | distillation column | (11) |
| 13: | valve | (—) |
| 14: | condensor | (12) |
| 15: | valve | (—) |
| 16: | valve | (—) |
| 17: | decanter | (13) |
| 18: | valve | (—) |
| 19: | valve | (—) |
| 20: | water feed | (14) |
| 21: | THFA feed | (15) |
| 22: | optional purge gas feed | (—) |
| 23: | crude reaction mixture | (—) |
| 24: | condensed reaction mixture | (—) |
| 25: | uncondensable gas(es) | (—) |
| 26: | crude degassed condensed reaction mixture | (16) |
| 27: | crude product | (—) |
| 28: | water | (17) |
| 29: | sump | (18) |
| 30: | head product (reflux) | (—) |
| 31: | condensed head product (reflux) | (—) |
| 32: | washed condensed head product (reflux) | (—) |
| 33: | water | (19) |
| 34: | product (DHP) | (20) |

FIG. 3 shows the reactor temperature measured during a 4.5 day run of a production process according to the present invention.

FIGS. 4 to 8 show the contents of different compounds in the crude product during a 4.5 day run of a production process according to the present invention (measured by gas chromatography with FID detection, column Type DB-1701 of dimensions 10 m×0.18 mm×0.4 μm, and expressed as % area of the peak of the respective compound in the chromatogram what corresponds with the weight % of the respective compound in the crude product), wherein

FIG. 4 shows the content of generated DHP in % area vs running time in days,

FIG. 5 shows the content of the by-product THP in % area vs running time in days,

FIG. 6 shows the content of the by-product cyclopentanone in % area vs. running time in days,

FIG. 7 shows the content of the by-product acrolein in % area vs. running time in days, and

FIG. 8 shows the content of unconverted THFA in % area vs. running time in days.

### DESCRIPTION OF THE INVENTION

A surprising feature of the present process for preparing DHP is that the catalyst is continuously regenerated in the presence of the water vapor. Thus, the catalyst has a long life and little regeneration, if ever, is required.

Typically, at the beginning of the process of the invention, THFA and water are fed separately through different lines for vaporization. At industrial scale, conveniently, waste heat may be used for preheating the starting compounds. In case waste heat is not sufficient to heat the starting compounds to the desired temperature, additional vaporizers may be used. Typically, the vaporized THFA is entrained in the water vapor immediately before entering the reactor in a mixing device (see FIG. 1 and Example below).

The weight ratio between THFA and water in the vapor phase when contacting the Al₂O₃-comprising catalyst is at least 1:10. Typically, the weight ratio of THFA to water vapor is in the range of 1:10 to 10:1. In another preferred embodiment the weight ratio of THFA to water vapor is in the range of 1:5 to 5:1, more preferably 1:2 to 2:1, in the latter case for example about 1:1.

Typically, the THFA is contacted with the solid phase catalyst by passing the THFA entrained in the carrier gas over/through the catalyst. In the Al₂O₃-comprising catalyst used according to the instant process, the Al₂O₃ may be used as the sole catalytically active component, or Al₂O₃ may be used in combination with one or more further catalytically active components. Examples of catalytically active components include further oxides such as TiO₂, V₂O₅ or MoO₃. The content of Al₂O₃ in the solid phase catalyst is typically within a range of from 50 to 100 weight % and preferably is at least 75 weight %, more preferably at least 90 weight %. Most preferably, the catalyst is essentially composed of Al₂O₃. Preferably, the η-modification of Al₂O₃, i.e., η-Al₂O₃, is used.

The THFA entrained in the water vapor typically is continuously passed through a heated reactor containing the Al₂O₃ catalyst. The residence time in the reactor is typically from 0.5 to 20 sec, preferably from 1 to 10 sec, more preferably from 1 to 5 sec, and most preferably from 1 to 2 sec.

In a preferred embodiment the DHP of the claimed use is obtained according to the present process, wherein the conversion of THFA on the Al₂O₃-comprising catalyst, is typically carried out at a temperature of 280 to 380° C., preferably of 300 to 350° C., for example at 330° C.

According to the present invention, a small amount of an inert gas such as nitrogen, argon, hydrogen, helium, carbon dioxide or a mixture thereof, may be optionally used in the conversion reaction. Nitrogen is the preferred additional gas in view of availability and price. Such additional gas may be used for example for safety reasons and/or to control the contact time of the reactants with the catalyst. In a preferred embodiment, the amount of nitrogen is typically in the range of 0.1 to 3 weight %, preferably of 0.5 to 2 weight % relative to the THFA/water vapor mixture passed over the catalyst.

In the reactor, the THFA vapor is converted to DHP vapor and water vapor. The obtained DHP and water vapors, together with the water vapor of the carrier gas, leaving the reactor are condensed and are typically passed through a separator or decanter where the water is removed from DHP.

The DHP obtained according to the invention may be subjected to further purification, to meet the standards required for the production of pharmaceutical compounds or agrochemicals. The most common by-products generated in DHP production are tetrahydropyran (THP), cyclopentanone and acrolein. Also some unreacted THFA is typically contained in the crude product. Further purification steps typically include distillation.

As THP, acrolein and cyclopentanone, typical and dominating by-products of the reaction, form azeotropic mixtures with the residual water of the crude product obtained after the decanter (no. 8 in FIG. 1, no. 9 in FIG. 2), said by-products may be removed from the raw product by azeotropic distillation. The azeotropic distillation may be carried out according to methods known in the art. In a further embodiment THP can be obtained in the inventive process by further purification of the waste water (33) from decanter (17).

For more efficient separation, the distillation is typically carried out by rectification. To remove the azeotropic mixtures, the reflux (not specified in FIG. 1, no. 28/29 and 30 of FIG. 2) may be passed partly or completely through a decanter (no. 13 in FIG. 1, no. 17 in FIG. 2) filled with water (i.e. washed), and where the azeotropic mixtures and the contaminants accumulate in said water, which is removed an replaced with fresh at suitable intervals.

The use of water vapor as carrier gas in the catalyzed production of DHP has the advantage that water and water vapor are abundantly available in industrial facilities. Moreover, after completion of the reaction, the water is easily condensed and separated from the crude DHP. The easy condensation and separation of the water also circumvents the problem of product loss. As the condensed water and the crude organic DHP are both in the liquid phase, their separation in a decanter is straightforward. Although the disposed water may still contain some DHP, the DHP loss is reduced compared to a process using large volumes of nitrogen as entrainer gas also which entrains a considerable amount of product.

Moreover, use of water vapor as a carrier gas requires less regeneration steps of the Al₂O₃-comprising catalyst compared to the state of the art. Without wishing to be bound by theory, it is believed that running the catalytic conversion with water vapor avoids catalyst regeneration to a large extent, as steam has similar effects as air and oxygen. The catalyst is thus continuously regenerated in the presence of water vapor. To run a DHP production truly continuous in the art it is recommended to have two catalytic reactor columns in parallel, thus, while one reactor is in use, the catalyst in the other reactor can be recycled. The present invention allows operating with only one reactor to be truly continuous. Furthermore, no waste products (such as organics, tar, carbon black) from regenerating the catalyst need to be released to the atmosphere without regenerating with an oxygen containing gas. A further advantage of the “continuous regeneration” is that there is no induction phase while restarting the reaction after regeneration. In the art such “induction phase” is a further reason for loss of product.

Since 3,4-dihydro-2H-pyran is an important building block in the synthesis of pharmaceutical and agrochemical compounds we further claim DHP obtained by the present process as well as the use of said DHP in industrial chemistry. Dihydropyran is commonly used to prepare tetrahydropyran (THP, CAS [142-68-7]) by hydrogenation in the presence of a catalyst, preferably a metal catalyst, for example Raney-Nickel, Raney-Cobalt, palladium or platinum. Thus, we also claim a process for the preparation of TFP, comprising hydrogenation of DHP in the presence of a catalyst, wherein the DHP is obtained by the instant process.

The present invention is illustrated in more detail by the following non-limiting examples.

### EXAMPLES

### Example 1

### Production of DHP Using H₂O Vapor as Carrier Gas

In this specific embodiment, the conversion of THFA to DHP was carried out in continuous mode and run for four days. References to FIG. 1 and FIG. 2 are indicated in brackets, such as (14/20) for water feed according to no. 14 in FIG. 1 and no. 20 in FIG. 2. Indications without a corresponding number are indicated with a two dashes, such as (--/8) for the gas-liquid separator which is not shown in FIG. 1. The reaction has been carried out in a ring-gap reactor of about 80 L internal volume, which has a treatable reaction tube having an internal length l of about 1700 mm, an internal diameter d¹ of about 436 mm in the interior of which is situated in an, if appropriate, rotatable cylindrical displacement body which extends over the entire length of the reaction tube, and is arranged axially symmetrically while having an outer diameter d² of about 500 mm.

Water (14/20) was pumped through heat exchanger (1/1) and preheated by the raw product stream leaving the reactor. After leaving the heat exchanger (1/1) the water had a temperature of about 195 to 210° C. The water vapor was further passed through vaporizers (2/2) and (3/3) to reach a temperature of about 370 to 395° C.

THFA (15/21) was pumped through heat exchanger (4/4) and then through vaporizer (5/5) to reach a temperature of 300° C. The gaseous THFA was entrained in the water vapor in a mixing device (6/6) and passed through a ring gap-reactor (7/7) charged with 49.3 kg of Al₂O₃ (N or Pro® Saint-Gobain, SA 3X77). The temperature in the reactor was 350° C. at the beginning of the production run and was further maintained at 330° C. The reactor pressure was maintained at atmospheric pressure. A small amount of nitrogen (1 kg/h) as a purge gas (--/22) was fed into the system between heat exchanger (1/1) and vaporizer (2/2). The feed of the THFA was 60 kg/h and the feed of the water was about 54 kg/h respectively (total THFA feed: 5.3 tons).

The residence time of the heated mixture in the reactor was approx. 1.3 sec and the WHSV (weight hourly space velocity, i.e. weight of feed flowing per unit weight of the catalyst per hour) was 2.3 h⁻¹.

The obtained crude reaction mixture (--/23) was condensed in the heat exchanger (1/1) and nitrogen and other uncondensable gases were removed in a separator (--/8). The crude degassed reaction mixture (16/26), mainly consisting of DHP and water, was further passed into a decanter (8/9) and separated from water. The water (17/28) was disposed as it contains at room temperature only about 1.6 weight % of DHP. Following separation from water, the crude product (--/27) contained 94 weight % of DHP, 1.8 weight % of THP, 1 weight % of THFA, 0.6 weight % of H₂O, 0.035 weight % of acrolein and 0.06 weight % of cyclopentanone and the yield was 82%.

An azeotropic distillation was carried out in batch mode. About 4.2 m³ of crude product were collected in tanks (9/10) after separation of water in the decanter before the crude product was subjected to distillation in two portions. A first part of the crude product was filled into a 3.2 m³ distillation boiler (10/11) and heated to reflux over a column (11/12) with a structured packing (Sulzer Mellapack 250 Y) having 11 theoretical plates. The distillation was carried out at atmospheric pressure. The sump (18/29) was periodically removed. The second part of the crude product was filled in the distillation boiler (10/11) when a respective amount from the first filling was distilled off.

At the beginning, the condensed head product (31) (reflux) was returned completely to column (11/12) with a rate of 900 L/h after passing a condenser (12/14). The decanter (13/17) was charged ⅔ with water, and ⅔ of the reflux was passed through the decanter before returning it to the column. The azeotropes remained in the decanter (13/17) and the water (19/33) in the decanter was replaced every 30 min until the organic phase met the required specifications, particularly until the acrolein content was 20 ppm or less. At that point, the ratio of reflux (i.e., about 600 L/h) and DHP removal (i.e. about 200 L/h) was set to about 3:1. The DHP (20/34) was distilled into one or more suitable tank(s). The distillation was interrupted when the temperature in the distillation boiler dropped to 130° C.

The purified DHP (20/34) thus obtained consisted of 97.77 weight % of DHP, 1.64 weight % of THP, 0.2 weight % of H₂O and 0.0017 weight % of acrolein as determined by gas chromatography. Cyclopentanone was below detection limits after distillation. DHP yield after distillation was 74%. It must be noted that in some distillation batches we obtained a very low acrolein level of only 6 to 9 ppm.

As can be seen from FIGS. 3 to 8, the conversion rate of THFA to DHP was consistently quantitative over four days. The amounts of generated by-products were low and did not increase over time. This demonstrates stable conversion with excellent yields when using water vapor as carrier gas. It is also evident that the process of the present invention may be carried out over long periods of time before regeneration of the catalyst is required. In the state of the art the life time of the catalyst in production corresponds to the recycle time of the catalyst. Under the conditions of the present invention even after 4.5 days of production the catalyst revealed no tendency of inactivation. Also, after 4.5 days by optical control the catalyst looked like fresh prepared catalyst and no deterioration of the catalyst could be observed.

Except for the start-up phase, the content of DHP in the crude product turned out to be very high and at least 94 weight % when using water vapor as carrier gas. On the other hand, the amounts of the by-products THP, cyclopentanone and acrolein in the crude product are surprisingly low. Typically, the amount of THP is below 2.0 weight %, the amount of acrolein is below 0.05 weight % and the amount of cyclopentanone is below 0.1 weight %. Further, the amount of unreacted THFA is typically below 1.5 weight % and the amount of residual water is typically below 1.0 weight %. Specifically, the amounts of cyclopentanone and acrolein are considerably lower than in industrial processes using nitrogen as carrier gas. Using water vapor as entrainer also leads to unexpectedly low content of acrolein in the DHP product.

### Example 2

### General Example for Use of DHP as Protecting Agent (Extracted from Alpagut Y., Inaugural Dissertation, Cologne, 2008)

A reaction flask is charged with phenol (12 g, 0.10 mol) and dihydropyran (10 mL, 0.11 mol) and cooled to about 0° C. Under stirring CF₃COOH (0.07 mL) is added slowly. The reaction mixture is stirred for 1 h at 0° C. and further stirred over night at room temperature. The obtained clear and colorless solution is washed three times each with 1 M NaOH solution and saturated NaCl solution. The combined organic phases are dried over K₂CO₃ and filtered. The filtrate is distilled at 0.9 mbar. About 8.4 g 2-phenyloxytetrahydropyran (0.05 mol) can be obtained at a boiling temperature of 77° C. as viscous colorless oil. The protection group can be cleaved off while stirring in an acetic acid/THF/water mixture for 4 h under reflux conditions.

Exact procedures for the formation of THP from DHP can be found in literature, for example in D. W. Andrus et al., Org. Synth. 1943, 23, 90.

Exact procedures for the formation of a compound of formula III, i.e. usage of DHP as a protecting agent can also be found in literature, for example in Alpagut Y., Inaugural Dissertation, Cologne, 2008 or in Greene, T. W. as cited above.

Because of the very low level of by-products of DHP obtained by the instant processes, said DHP is better suited for further reaction steps without purification then DHP obtained according to the state of the art. According to the above, use of DHP obtained by the instant process includes the preparation of a compound of formula III as well as the release of an eventually different alcohol, thiol, acid, amine or amide after acidic ring opening then being used for preparing the tetrahydropyranyl derivative of formula III.

1. A process for the protection of a compound having at least one reactive functional group selected from hydroxy, mercapto, carboxy, amino and amido, with 3,4-dihydro-2H-pyran (DHP), characterized in that said DHP is obtained by contacting tetrahydrofurfuryl alcohol (THFA) entrained in a carrier gas with a catalyst comprising aluminium oxide (Al₂O₃), and wherein the carrier gas is water vapor.

2. The process of claim 1, wherein the compound having at least one reactive functional group is an oligomer, a polymer or is a discrete compound.

3. The process of claim 2, wherein the compound having at least one reactive functional group is of formula
H—X III,
wherein
X is selected from −OR¹, —SR¹, and —OC(═O)R¹, wherein R¹ is a substituent selected from the group consisting of C₁₋₁₀-alkyl, C₂₋₁₁-acyl, C₃₋₈-cycloalkyl, C₄₋₉-cycloalkanoyl, aryl, aralkyl, aroyl, hetaryl and hetaroyl, or
X is —NR²R³, wherein R² and R³ independently are selected from the group consisting of hydrogen, C₁₋₁₀-alkyl, C₂₋₁₁-acyl, C₃₋₈-cycloalkyl, C₄₋₉-cycloalkanoyl, aryl, aralkyl, aroyl, hetaryl and hetaroyl, with the proviso that at least one of R² and R³ is different from hydrogen, and wherein the reaction product is of formula 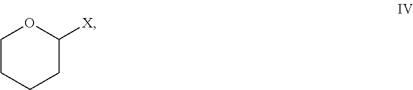
wherein X is as defined above.

4. The process of claim 1, wherein the catalyst in the process for preparing DHP is continuously regenerated in the presence of the water vapor.

5. The process of claim 1, wherein the weight ratio of THFA to water vapor in the process for preparing DHP is at least 1:10.

6. The process of claim 5, wherein the weight ratio of THFA to water vapor is in the range of 1:10 to 10:1, more preferably in the range 1:5 to 5:1.

7. The process of claim 1, wherein the catalyst further comprises one or more catalytically active components in addition to Al₂O₃.

8. The process of claim 7, wherein additional catalytically active components comprises a catalytically active oxide selected from the group consisting of TiO₂, V₂O₅ and MoO₃.

9. The process of claim 1, wherein the synthesis of DHP is carried out at a temperature of from 280 to 380° C., preferably of from 300 to 350° C.

10. The process of claim 1, wherein DHP is subjected to a purification step, preferably to an azeotropic distillation before use as a protective agent.
